(19) United States
(12) Patent Application Publication
(10) Pub. No.: US 2008/0281100 A1
(43) Pub. Date: Nov. 13, 2008

(54) Pure paliperidone and processes for preparing thereof

(76) Inventors: Santiago INI, Haifa (IL); Naama CHASID, Petah Tikva (IL); Kobi CHEN, Kfar-Saba (IL); Osnat PORTER-KLEKS, Petach-Tikva (IL)

Correspondence Address: KENYON & KENYON LLP, ONE BROADWAY, NEW YORK, NY, 10004, US

(21) Appl. No.: 12/070,130

(22) Filed: Feb. 14, 2008

Related U.S. Application Data
(63) Continuation of application No. 11/889,558, filed on Aug. 14, 2007.
(60) Provisional application No. 60/963,922, filed on Aug. 7, 2007.
(60) Provisional application No. 60/928,745, filed on May 10, 2007.
(60) Provisional application No. 60/935,093, filed on Jul. 26, 2007.
(60) Provisional application No. 60/928,747, filed on May 10, 2007.
(60) Provisional application No. 60/930,392, filed on May 15, 2007.
(60) Provisional application No. 60/929,126, filed on Jun. 14, 2007.
(60) Provisional application No. 60/958,571, filed on Jul. 5, 2007.
(60) Provisional application No. 60/929,703, filed on Jul. 10, 2007.
(60) Provisional application No. 60/935,094, filed on Jul. 26, 2007.

(51) Int. Cl. C07D 487/04

(52) U.S. Cl. 544/282

(57) ABSTRACT

The present invention provides pure paliperidone comprising less than about 0.1%, preferably less than about 0.05% and more preferably less than about 0.02%, impurity X as well as purification processes to obtain thereof.

### CROSS REFERENCE TO RELATED APPLICATIONS

This patent application claims the benefits of U.S. Provisional Application No. 60/963,922 filed on Aug. 7, 2007, No. 60/928,745 filed May 10, 2007, No. 60/935,093 filed Jul. 26, 2007, No. 60/928,747 filed May 10, 2007, No. 60/930,392 filed May 15, 2007, No. 60/929,126 filed Jun. 14, 2007, No. 60/958,571 filed Jul. 5, 2007, No. 60/929,703 filed Jul. 10, 2007, and No. 60/935,094 filed Jul. 26, 2007 and claims the benefit of U.S. Non-Provisional application Ser. No. 11/889,558 filed Aug. 14, 2007, the disclosures of which are hereby incorporated by reference, wherein this patent application is a continuation-in-part application of U.S. Non-Provisional application Ser. No. 11/889,558.

### FIELD OF INVENTION

The present invention relates to a process for the purification of Paliperidone (“PLP”) from its impurities. Also, the present invention relates to pure paliperidone.

### BACKGROUND

Paliperidone, 3-[2-[4-(6-fluorobenzo[d]isoxazol-3-yl)-1-piperidyl]ethyl]-7-hydroxy-4-methyl-1,5-diazabicyclo[4.4.0]deca-3,5-dien-2-one, is a 5-HT antagonist belonging to the chemical class of benzisoxazole derivatives and a racemic mixture having the following structural formula:

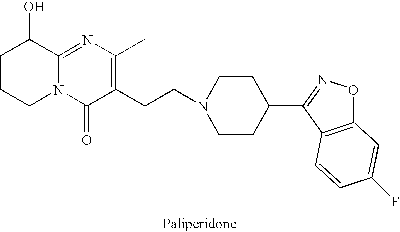

Paliperidone is a metabolite of Risperidone. Marketed under the name, Invega®, Paliperidone is a psychotropic agent approved in the United States for the treatment of schizophrenia.

Processes for the synthesis of Paliperidone, are described in U.S. Pat. No. 5,158,952. Another process for the synthesis of a precursor of Paliperidone, (3-(2-chloroethyl)-2-methyl-9-benzyloxy-4H-pyrido[1,2-a]-pyrimidine-4-one), is described in the above publications.

Like any synthetic compound, paliperidone can contain extraneous compounds or impurities that can come from many sources. They can be unreacted starting materials, by-products of the reaction, products of side reactions, or degradation products. Impurities in paliperidone or any active pharmaceutical ingredient (API) are undesirable and, in extreme cases, might even be harmful to a patient being treated with a dosage form containing the API.

It is also known in the art that impurities in an API may arise from degradation of the API itself, which is related to the stability of the pure API during storage, and the manufacturing process, including the chemical synthesis. Process impurities include unreacted starting materials, chemical derivatives of impurities contained in starting materials, synthetic by-products, and degradation products.

In addition to stability, which is a factor in the shelf life of the API, the purity of the API produced in the commercial manufacturing process is clearly a necessary condition for commercialization. Impurities introduced during commercial manufacturing processes must be limited to very small amounts, and are preferably substantially absent. For example, the International Conference on Harmonization of Technical Requirements for Registration for Human Use (“ICH”) Q7A guidance for API manufacturers requires that process impurities be maintained below set limits by specifying the quality of raw materials, controlling process parameters, such as temperature, pressure, time, and stoichiometric ratios, and including purification steps, such as crystallization, distillation, and liquid-liquid extraction, in the manufacturing process.

The product mixture of a chemical reaction is rarely a single compound with sufficient purity to comply with pharmaceutical standards. Side products and by-products of the reaction and adjunct reagents used in the reaction will, in most cases, also be present in the product mixture. At certain stages during processing of the API, paliperidone, it must be analyzed for purity, typically, by HPLC, TLC or GC analysis, to determine if it is suitable for continued processing and, ultimately, for use in a pharmaceutical product. The API need not be absolutely pure, as absolute purity is a theoretical ideal that is typically unattainable. Rather, purity standards are set with the intention of ensuring that an API is as free of impurities as possible, and, thus, are as safe as possible for clinical use. As discussed above, in the United States, the Food and Drug Administration guidelines recommend that the amounts of some impurities be limited to less than 0.1 percent.

Generally, side products, by-products, and adjunct reagents (collectively “impurities”) are identified spectroscopically and/or with another physical method, and then associated with a peak position, such as that in a chromatogram, or a spot on a TLC plate. (Strobel p. 953, Strobel, H. A.; Heineman, W. R., Chemical Instrumentation: A Systematic Approach, 3rd dd. (Wiley & Sons: New York 1989)). Thereafter, the impurity can be identified, e.g., by its relative position in the chromatogram, where the position in a chromatogram is conventionally measured in minutes between injection of the sample on the column and elution of the particular component through the detector. The relative position in the chromatogram is known as the “retention time.”

The retention time can vary about a mean value based upon the condition of the instrumentation, as well as many other factors. To mitigate the effects such variations have upon accurate identification of an impurity, practitioners use the “relative retention time” (“RRT”) to identify impurities. (Strobel p. 922). The RRT of an impurity is its retention time divided by the retention time of a reference marker. It may be advantageous to select a compound other than the API that is added to, or present in, the mixture in an amount sufficiently large to be detectable and sufficiently low as not to saturate the column, and to use that compound as the reference marker for determination of the RRT.

Two potential impurities of paliperidone are: 3-[2-[4-(6-fluorobenzo[d]isoxazol-3-yl)-1-oxypiperidin-1-yl]ethyl]-7-hydroxy-4-methyl-1,5-diazabicyclo[4.4.0]deca-3,5-dien-2-one (PLP-NO) and 2-[4-(6-fluoro-1,2-benzisoxazol-3-yl)piperidin-1-carboxylicacid]-7-hydroxy-2-methyl-6,7,8,9-tetrahydro-4H-pyrido[1,2-a]pyrimidin-4-one-3-yl-ethyl ester (PLP-car):

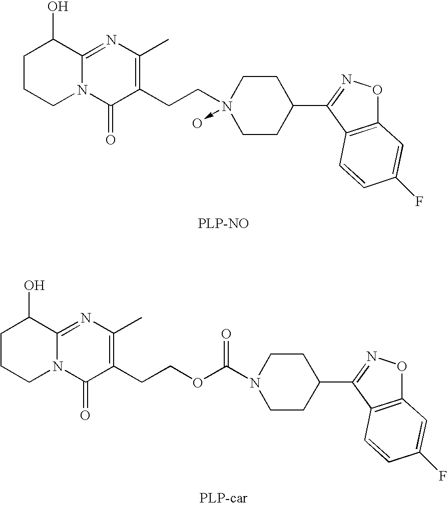

These impurities remain in the final product.

Additionally, the commercial tablet Invega® appears to contain 0.10% of PLP-NO.

There is a need in the art for paliperidone having a higher purity, as well as purification processes for obtaining thereof.

### SUMMARY OF THE INVENTION

In one embodiment, the present invention provides paliperidone containing less than about 0.1% of impurity X. Preferably the paliperidone of the present invention contains less than about 0.05% and more preferably less than about 0.02% of the impurity X.

In another embodiment, the present invention provides paliperidone having a total purity of at least about 98%. Preferably, the total purity is at least about 99%, most preferably at least about 99.9%.

In another embodiment, the present invention provides processes for purifying paliperidone.

### BRIEF DESCRIPTION OF THE DRAWINGS

FIG. 1 shows a typical chromatogram from an analysis of a paliperidone sample of the present invention performed using the HPLC method disclosed herein, wherein the unit for the horizontal axis is minute.

FIG. 2 shows the data obtained in the HPLC analysis resulting in the chromatogram of FIG. 1.

### DETAILED DESCRIPTION OF THE INVENTION

As used herein, the term “CMHTP” refers to 3-(2-chloroethyl)-6,7,8,9-tetrahydro-9-hydroxy-2-methyl-4H-pyrrido[1,2-a]-pyrimidin-4-one of the following structure:

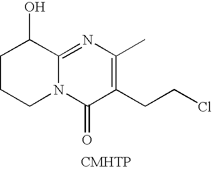

As used herein, the term “FBIP” refers to 6-fluoro-3-piperidino-1,2-benisoxazole of the following structure:

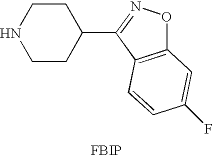

As used herein, the term “PLP-NO” refers to 3-[2-[4-(6-fluorobenzo[d]isoxazol-3-yl)-1-oxypiperidin-1-yl]ethyl]-7-hydroxy-4-methyl-1,5-diazabicyclo[4.4.0]deca-3,5-dien-2-one of the following structure:

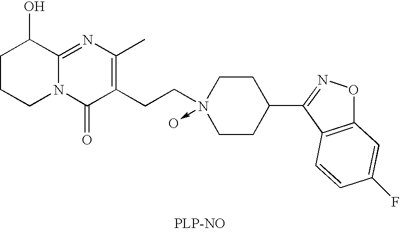

As used herein, the term “PLP-car” refers to PLP carbamate or paliperidone carbamate, i.e., 2-[4-(6-fluoro-1,2-benzisoxazol-3-yl)piperidin-1-carboxylicacid]-7-hydroxy-2-methyl-6,7,8,9-tetrahydro-4H-pyrido[1,2-a]pyrimidin-4-one-3-yl-ethyl ester of the following structure:

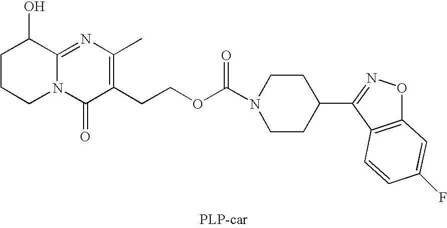

As used herein, the term “impurity X” refers to a potential impurity of paliperidone having a relative retention time (“RRT”) of about 1.27, as relative to the retention time of paliperidone, based on the HPLC method described in the Examples below.

As used herein, the term “reduced pressure” refers to a pressure of under 100 nun Hg.

In the present application, the term “room temperature” means a temperature of about 20 oC to about 25 oC.

As used herein, the term “slurrying” means stirring a mixture of a solid in a liquid, e.g., stirring a suspension of solid powder in a liquid.

As used herein, the term “N/D” represents none detected.

The present invention provides pure paliperidone, as well as processes for preparing thereof. As used herein, “pure paliperidone” refers to paliperidone containing less than about 0.1% of the impurity X. Preferably the paliperidone of the present invention contains less than about 0.05% and more preferably less than about 0.02% of the impurity X. The purity is preferably measured by HPLC, and is presented as % area as shown in the HPLC chromatogram.

The pure paliperidone of the present invention has a total purity of at least about 98%. Preferably, the total purity is at least about 99%, most preferably at least about 99.9%. For example, the total purity of the pure paliperidone of the present invention can be about 98% to about 99.95%, about 98% to about 99.99%, about 99% to about 99.95%, or about 99% to about 99.99%. The purity is preferably measured as described above.

The present invention further provides a process for preparing the pure paliperidone via the purification of paliperidone. This process comprises crystallizing paliperidone from a solvent selected from the group consisting of: C3-6 ketone or a mixture thereof with water, N-methylpyrrolidone, C3-6 amides, halo-substituted C6-12 aromatic hydrocarbons propylene glycole, dim ethyl sulfoxide, di-methyl carbonate, C1-4 alkyl alcohols, a mixture of a C1-8 alkyl alcohol and water, acetonitrile or a mixture thereof with water, C2-6 alkyl acetates or their mixture with water, cellosolve, dimethyl carbonate, polyethylene glycol methyl ether and C2-8 ethers. The crystallization is preferably performed by dissolving paliperidone in the above solvent, preferably by heating the reaction mixture to allow complete dissolution, followed by cooling of the obtained solution, whereby paliperidone crystallizes. Preferred C3-6 ketones are acetone, methyl ethyl ketone (MEK) and methyl iso-butyl ketone (MIBK). Preferred C3-6 amides are dimethylacetamide and dimethylformamide. Preferred halo-substituted C6-12 aromatic hydrocarbons are chlorobenzene and dichlorobenzene. Preferred C1-4 alkyl alcohols are methanol, ethanol, n-propanpl, isopropanol, n-butanol, isobutanol and 2-butanol. Preferred C2-6 alkyl acetates are ethyl acetate and isobutyl acetate. Preferred C2-8 ethers are dibutyl ether and polyethylene glycol (PGME). Most preferably, the solvent is a mixture of acetone and water. When a mixture is used (such as acetone:water, ethanol:water etc.), the ratio between the solvents is between about 1:1 to about 3:1 by volume. The ratio of acetone to water is preferably about 3:1 by volume. Following crystallization, the obtained product is preferably recovered by filtering, washing of the obtained crystals, and drying, preferably overnight under reduced pressure.

Paliperidone obtained by the above process preferably contains impurity X in an amount of less than about 0.1% and PLP-car in an amount of less than about 0.2%, and more preferably impurity X in an amount of less than about 0.05% and PLP-car in an amount less than about 0.1%. The above crystallization process may be repeated in order to further purify the obtained paliperidone, so that the impurity X and PLP-car levels may be reduced to less than about 0.02%.

The total purity of the paliperidone obtained by the above processes is of at least about 98%, more preferably, at least about 99% and most preferably at least about 99.9%. Preferably, the purity is measured as described above.

The present invention provides a process for preparing pure paliperidone via the purification of paliperidone comprising crystallizing paliperidone by combining a solution of paliperidone in a first solvent with an anti-solvent. Preferably, the solution is obtained by dissolving paliperidone in dichloromethane, preferably at a reflux temperature. The obtained solution is then cooled, preferably to a temperature of about 0° C. to about 30° C., preferably to a temperature of about 20° C. to about 30° C., and most preferably of about 25° C., followed by admixing with the anti-solvent described above. The admixing may be done in any order, for example, the anti-solvent may be added to the solution, or alternatively, the solution may be added to the anti-solvent. When the hot solution is added to the anti-solvent, the temperature difference causes the fast crystallization. The addition may be added dropwise or in one volume. Preferably the first solvent is selected from the group consisting of: dichloromethane, dioxane and C1-4 alkyl alcohols. Most preferably the first solvent is selected from the group consisting of: dichloromethane, dioxane, butanol and n-propanol. Preferably, the anti-solvent is selected from the group consisting of C3-6 ketones, C3-6 ethers, acetonitrile, C3-7 straight and cyclic carbohydrates, C6-12 aromatic carbohydrates and water. More preferably, the anti-solvent is selected from the group consisting of: methyl t-butyl ether (MTBE), MEK, acetone, MIBK, acetonitrile, cyclohexane, hexane, heptane, toluene, benzene, xylene and water. Even more preferably, the anti solvent is selected from the group consisting of MTBE, MEK, acetonitrile, cyclohexane, heptane, toluene and water. Most preferably, the anti-solvent is selected from the group consisting of acetonitrile, MEK, toluene and MTBE. The obtained mixture is then preferably maintained for at least about 5 minutes or till crystallization occurs, more preferably between about 5 minutes and about 6 hours, most preferably for about 1.5 hours, and preferably under stirring. The obtained product is preferably recovered by filtering.

Paliperidone obtained by the above process preferably contains impurity X in an amount of less than about 0.1% (preferably less than about 0.05%) and PLP-car in an amount of less than about 0.04%. The above crystallization process may be repeated in order to further purify the obtained paliperidone, so that the impurity X and PLP-car levels may be reduced to less than about 0.02%.

The total purity of the paliperidone obtained by the above processes is of at least about 98%, more preferably, at least about 99% and most preferably at least about 99.9%. Preferably, the purity is measured as described above.

The present invention provides a process for preparing pure paliperidone via the purification of paliperidone comprising slurrying paliperidone in an organic solvent. Preferably the slurrying is performed at a temperature of about 20° C. to about 70° C., more preferably at a temperature of about 25° C. to about 65° C. Preferably, the slurrying is performed for a period of time sufficient for purifying paliperidone, more preferably from about 30 minutes to about 24 hours. Preferably the organic solvent is selected from C1-4 alkyl alcohols, C3-5 ketones and water. Preferably the organic solvent is selected from ethanol, methanol, isopropanol, acetone and water. The obtained product is preferably recovered by filtering.

Paliperidone obtained by the above process preferably contains impurity X in an amount of less than about 0.1% (preferably less than about 0.05%), and PLP-Car in an amount of less than about 0.04%. The above slurrying process may be repeated in order to further purify the obtained paliperidone, so that the impurity X and PLP-car levels may be reduced to less than about 0.02%.

The total purity of the paliperidone obtained by the above processes is of at least about 98%, more preferably, at least about 99% and most preferably at least about 99.9%. Preferably, the purity is measured as described above.

The present invention further provides a process for preparing pure paliperidone via the purification of paliperidone comprising providing a paliperidone solution containing more than about 0.1% X or more than about 2% of any other impurity; admixing the solution with finely powdered carbon; and filtrating the admixture to obtain pure paliperidone. The filtering step is performed in order to remove the finely powdered carbon. Preferably the solution is obtained by dissolving paliperidone in an organic solvent. The organic solvent is preferably a mixture of acetone:water. Preferably, finely powdered carbon is an active carbon. The active carbon is preferably selected from the group consisting of HB ultra, CGP super, GBG, SX plus, ROX 0.8 and A super eur. The filtration is preferably done through hi-flow.

Paliperidone obtained by the above process preferably contains impurity X in an amount of less than about 0.1% (preferably less than about 0.05%) and PLP-car in an amount of less than about 0.05%. The above crystallization process may be repeated in order to further purify the obtained paliperidone, so that the X and PLP-car levels may be reduced to less than about 0.02%.

The total purity of the paliperidone obtained by the above processes is of at least about 98%, more preferably, at least about 99% and most preferably at least about 99.9%. Preferably, the purity is measured as described above.

The present invention also directs to the pure paliperidone prepared by any one of the paliperidone purification processes described above.

Having described the invention with reference to certain preferred embodiments, other embodiments will become apparent to one skilled in the art from consideration of the specification. The invention is further defined by reference to the following examples describing in detail the purification of paliperidone. It will be apparent to those skilled in the art that many modifications, both to materials and methods, may be practiced without departing from the scope of the invention.

### EXAMPLES

HPLC Method:

| Column & Packing: | Zorbax SB-Phenyl 250 × 4.6 mm, 5μ Part No: | | |
|---|---|---|---|
| | 880975-912 | | |
| Buffer: | 0.04M KH₂PO₄ pH 2.0 adjusted with H₃PO₄ | | |
| Eluent A: | 85% Buffer: 15% Acetonitrile | | |
| Eluent B: | 65% Buffer: 35% Acetonitrile | | |
| Gradient: | Time | % Eluent A | % Eluent B |
| | 0 | 100 | 0 |
| | 20 | 100 | 0 |
| | 21 | 100 | 0 |
| | 40 | 0 | 100 |
| | 60 | 0 | 100 |

| | |
|---|---|
| Flow | 1 mL/min |
| Run time: | 60 min |
| Equilibrium time: | 10 min |
| Sample volume: | 20 μL |
| Detector: | 238 nm |
| Column | 25° C. |
| temperature: | |
| Diluent | Eluent A |

### Sample Solution Preparation

Weigh accurately about 10 mg Paliperidone sample into a 10 mL volumetric flask, add 1 mL acetonitrile, sonicate until no chunks are observed (a few minutes) and dilute to volume with diluent.

### Calculation

Calculate the amount of unknown impurities as follows:

$$\% impurity i = \frac{area impurity i in samp . \times 100}{\sum ( area of all peaks )}$$

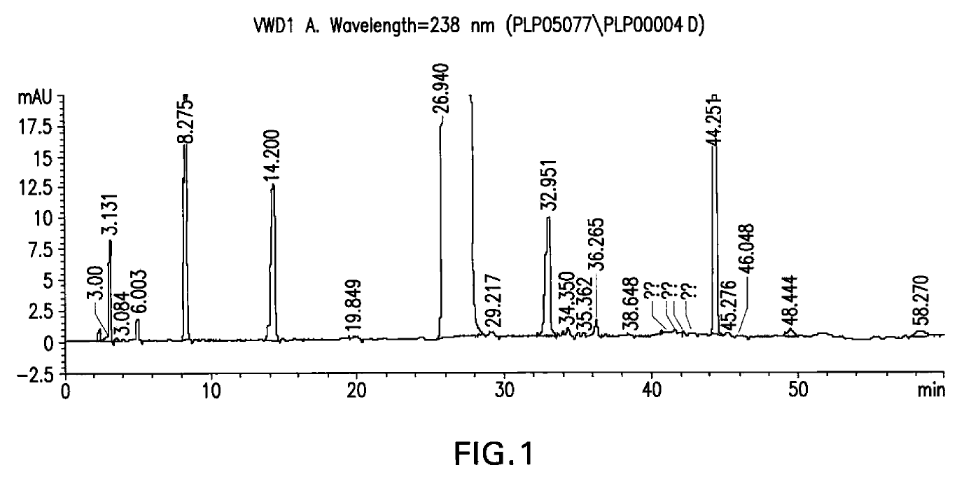

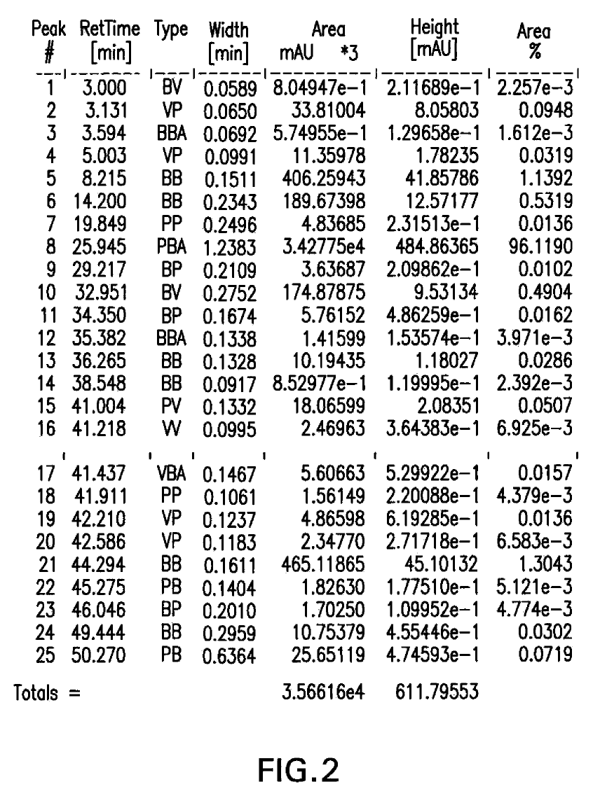

A typical chromatogram of the analysis of a paliperidone sample of the present invention obtained using the above HPLC method is shown in FIG. 1, with the HPLC data shown in FIG. 2. The typical retention times and relative retention times (relative to paliperidone) of the compounds resolved by the HPLC method are listed in the table below.

| Compound | Retention Time (min.) | RRT |
|---|---|---|
| CMHTP | 8,.2 | 0.32 |
| FBIP | 14.2 | 0.55 |
| PLP | 25.9 | 1.0 |
| Impurity X | 33.0 | 1.27 |
| PLP-CAR | 44.3 | 1.71 |

### Example 1

### Purification of Paliperidone from Impurity X by Crystallization

A slurry of paliperidone contaminated with X, in the indicated solvent, at the indicated volumes was heated to the indicated temperatures until complete dissolution, wherein each of the ratios presented in the table below represents volume ratio of the two solvents named immediately preceding the ratio. After the compound was dissolved, the oil bath was removed and the solution was cooled to room temperature (excepted where is indicated). The solid was filtrated and analyzed as shown in the next table.

| | Volumes | | | |
|---|---|---|---|---|
| | of | | X before | X after |
| | solvent | Heating | Crystallization | Crystallization |
| Solvent | (ml/g) | temp. | (%) | (%) |
| acetone | 155 | reflux | 0.53 | 0.27 |
| NMP | 21 | 65° C. | 0.53 | 0.19 |
| Acetone/water | 25 | reflux | 0.41 | 0.22 |
| (3:1) | | | | |
| ethanol | 80 | 70° C. | 0.41 | 0.32 |
| NMP¹ | 21 | 65° C. | 0.41 | 0.23 |
| Acetone/water | 40 | reflux | 0.67 | 0.35 |
| (3:1)¹ | | | | |

¹Cooled to 0° C.

### Example 2

### Preparation of Paliperidone Free of Impurity X

A slurry of 28 g Paliperidone (containing 0.26% of X) in a 1120 ml of a mixture of acetone/water (3:1) was heated to reflux till complete dissolution. After one hour, the solution was cooled to 0-4° C., filtrated, and washed with 60 ml. of acetone. The procedure was repeated three times and finally the material was dried in a vacuum oven at 50 oC under reduced pressure for overnight to give 15.2 g of Paliperidone containing less than 0.02% of X.

### Example 3

### Purification of Paliperidone from Impurity X by Addition of a Different Solvent

A slurry of Paliperidone (containing 0.41% of X) in 20 volumes (ml/g) of dichloromethane was heated to reflux until complete dissolution. The solution was cooled to room temperature and the indicated anti-solvent was gradually added until precipitation. The mixture was stirred at room temperature for 1.5 h and the solid was collected by vacuum filtration, and analyzed as shown in the next table.

| | Volumes of anti- | X after |
|---|---|---|
| Anti-solvent | solvent (ml/g) | Crystallization (%) |
| MTBE | 15 | 0.26 |
| MEK | 20 | 0.20 |
| Acetonitrile | 25 | 0.17 |
| Cyclohexane | 30 | 0.24 |
| heptane | 15 | 0.25 |
| toluene | 15 | 0.24 |

### Example 4

### Purification of Paliperidone from Impurity X by Slurrying in Different Solvents

A slurry of Paliperidone (containing 0.41% of X) in the indicated volume of one of the indicated solvents was stirred at the indicated temperatures and the indicated times, as indicated in the next table. The solid was collected by vacuum filtration and analyzed. The results are displayed in the next table.

| | | | | | X after |
|---|---|---|---|---|---|
| | Volumes of | Stirring | Stirring | | Crystallization |
| Solvent | solvent (ml/g) | temp. | time | | (%) |
| ethanol | 10 | 65° C. | 35 | min | 0.30 |
| methanol | 5 | 60° C. | 1 | h | 0.29 |
| methanol | 5 | room | 1 | h | 0.34 |
| | | temperature | | | |

### Example 5

### Purification of Paliperidone from Impurity X by Addition of a Different Solvent at a Different Temperature

A slurry of Paliperidone (containing 0.41% of X) in 7 volumes (ml/g) of one of the solvents indicated in the next table was heated to reflux until complete dissolution. The cooled anti-solvent as indicated in the next table was added at once. The resulting solid was collected by vacuum filtration, and analyzed as shown in the next table.

| | Anti- | Volumes of anti- | X after |
|---|---|---|---|
| Solvent | solvent | solvent (ml/g) | Crystallization (%) |
| dioxane | water | 15 | 0.35 |
| butanol | water | 70 | 0.39 |

### Example 6

### Purification of Paliperidone from Impurity X by Addition of a Different Hot Solvent

Slurry of Paliperidone (containing 0.41% of X) in the indicated solvent was heated to reflux until complete dissolution. The hot solution was added dropwise into an anti-solvent that was previously cooled in an ice bath. The resulting solid was collected by vacuum filtration, and analyzed as shown in the next table.

| | Volumes | | Volumes | X after |
|---|---|---|---|---|
| | of solvent | Anti- | of anti- | Crystallization |
| Solvent | (ml/g) | solvent | solvent (ml/g) | (%) |
| n-propanol | 30 | water | 50 | 0.24 |
| dichloromethane | 17 | hexane | 50 | 0.33 |
| dioxane | 10 | water | 50 | 0.35 |

### Example 7

### Purification of Paliperidone from Impurity X by Filtration through Activated Carbon

A slurry of paliperidone (contaminated with 0.67% X) in 40 volumes (i.e., g/40 ml) of acetone/water (3:1, volume ratio) was heated to reflux until complete dissolution. After the compound was dissolved, the hot solution was filtrated through hi-flow and cooled in an ice bath. The solid was filtrated and analyzed as shown in the next table.

| | X after |
|---|---|
| Type of active carbon | Crystallization (%) |
| HB ultra | 0.16 |
| CGP super | 0.42 |
| GBG | 0.24 |
| SX plus | 0.24 |
| ROX 0.8 | 0.29 |
| A super eur | 0.12 |

### Example 8

### Purification of Paliperidone from PLP-Car by Crystallization

A slurry of paliperidone contaminated with PLP-Car, in one of the solvents indicated in the next table, at the indicated volume was heated to the indicated temperatures until complete dissolution, wherein each of the ratios presented in the table below represents volume ratio of the two solvents named immediately preceding the ratio. After the compound was dissolved, the oil bath was removed and the solution was cooled to room temperature (except where is indicated). The solid was filtrated and analyzed as shown in the next table.

| | Volumes | | | |
|---|---|---|---|---|
| | of solvent | Heating | PLP-car before | PLP-car after |
| Solvent | (ml/g) | temp. | Crystallization (%) | Crystallization (%) |
| DMF | 5 | reflux | 1.51 | N/D |
| Dimethyl acetamide | 5 | reflux | 1.51 | 0.16 |
| Dichlorobenzene | 5 | reflux | 1.51 | 0.63 |
| Propylene glycole | 5 | reflux | 1.51 | 0.92 |
| DMSO | 5 | reflux | 1.51 | 0.38 |
| Acetone/water 3:1 | 40 | reflux | 1.51 | 0.44 |
| DMC | 33 | reflux | 1.51 | 0.25 |
| 2-butanol | 20 | reflux | 1.51 | 0.51 |
| MIPK | 54 | reflux | 1.51 | 0.37 |
| Iso-butanol | 26 | reflux | 1.51 | 0.57 |
| NMP | 5 | 140° C. | 1.51 | N/D |
| Ethanol/water 3:1 | 12 | reflux | 1.51 | 0.76 |
| MEK | 69 | reflux | 1.51 | 0.22 |
| acetonitrile | 100 | reflux | 1.51 | 0.21 |
| EtOAc/water 3:1 | 50 | reflux | 1.51 | 0.21 |
| acetone | 155 | reflux | 1.51 | 0.17 |
| Acetonitrile/water 1:1 | 40 | reflux | 1.31 | 0.79 |
| n-butanol | 23 | 135° C. | 1.31 | 0.45 |
| cellosolve | 8 | 115° C. | 1.31 | 0.33 |
| chlorobenzene | 7 | 115° C. | 1.31 | 0.36 |
| DMSO | 5 | 110° C. | 1.31 | 0.22 |
| dichlorobenzene | 5 | 120° C. | 1.31 | 0.56 |
| Propylene glycol | 7 | 120° C. | 1.31 | 0.60 |
| Dibutyl ether | 140 | 130° C. | 1.31 | 1.07 |
| PGME | 7 | 130° C. | 1.31 | 0.32 |
| Iso-butyl acetate | 35 | reflux | 1.31 | 0.45 |
| n-propanol | 30 | 90° C. | 1.31 | 0.48 |
| ethanol | 80 | 70° C. | 1.31 | 0.68 |
| Acetone/water (3:1)¹ | 40 | reflux | 1.31 | 0.09 |
| IPA/water (1:1)¹ | 19 | reflux | 0.57 | 0.07 |
| Methanol/water (3:1)¹ | 37 | reflux | 0.57 | 0.10 |

### Example 9

### Purification of Paliperidone from PLP-Car by Addition of a Different Solvent

A slurry of Paliperidone (containing 1.31% of PLP-Car) in 20 volumes (ml/g) of dichloromethane was heated to reflux until complete dissolution. The resulting solution was cooled to room temperature and one of the anti-solvents indicated in the next table was gradually added until precipitation. The mixture was stirred at room temperature for 1.5 h and the solid was collected by vacuum filtration, and analyzed as shown in the next table.

| | Volumes of | |
|---|---|---|
| | anti-solvent | PLP-Car after |
| Anti-solvent | (ml/g) | Crystallization (%) |
| MTBE | 15 | 0.17 |
| MEK | 20 | 0.16 |
| Acetonitrile | 25 | 0.12 |
| Cyclohexane | 30 | 0.28 |
| heptane | 15 | 0.18 |
| toluene | 15 | 0.09 |

### Example 10

### Purification of Paliperidone from PLP-Car by Slurrying in Different Solvents

A slurry of Paliperidone in the indicated volume of one of the indicated solvents was stirred at the indicated temperature and the indicated time as shown in the next table. The solid was collected by vacuum filtration and analyzed. The results are displayed in the next table.

| | | | | PLP-CAR | PLP-CAR |
|---|---|---|---|---|---|
| | Volumes | | | before | after |
| | of solvent | Stirring | Stirring | Crystal- | Crystal- |
| Solvent | (ml/g) | temp. | time | lization (%) | lization (%) |
| Ethanol | 10 | Room | 40 min | 1.31 | 1.15 |
| | | Temp. | | | |
| Ethanol | 10 | 65° C. | 35 min | 1.31 | 0.77 |
| Acetone | 10 | 60° C. | 17 h | 0.13 | N/D |
| IPA | 10 | 60° C. | 17 h | 0.13 | N/D |
| Acetone | 10 | 60° C. | 24 h | 1.31 | 0.47 |
| IPA | 10 | 60° C. | 24 h | 1.31 | 0.74 |
| Water | 10 | 60° C. | 25 h | 1.31 | 1.20 |
| Acetone | 10 | Room | 47 h | 0.13 | 0.04 |
| | | Temp. | | | |
| IPA | 10 | Room | 47 h | 0.13 | 0.04 |
| | | Temp. | | | |
| Methanol | 5 | 60° C. | 1 h | 1.31 | 0.89 |
| Methanol | 5 | Room | 1 h | 1.31 | 1.08 |
| | | Temp. | | | |

### Example 11

### Purification of Paliperidone from PLP-Car by Addition of a Different Solvent at a Different Temperature

A slurry of Paliperidone (containing 1.31% of PLP-Car) in 7 volumes (ml/g) of one of the solvents indicated in the next table was heated to reflux until complete dissolution. The cooled anti-solvent (cooled in an ice bath) in the volume indicated in the next table was added at once. The resulting solid was collected by vacuum filtration, and analyzed, wherein the analytical results are as shown in the next table.

| | Anti- | Volumes of anti- | PLP-Car after |
|---|---|---|---|
| Solvent | solvent | solvent (ml/g) | Crystallization (%) |
| dioxane | water | 15 | 0.69 |
| toluene | water | 35 | 1.18 |
| butanol | water | 70 | 0.04 |

### Example 12

### Purification of Paliperidone from PLP-Car by Addition of a Different Hot Solvent

A slurry of Paliperidone (containing 1.31% of PLP-Car) in one the solvents indicated in the next table was heated to reflux until complete dissolution. The hot solution was added dropwise into the indicated anti-solvent that was previously cooled in an ice bath. The resulting solid was collected by vacuum filtration, and analyzed, wherein the analytical results are as shown in the next table.

| | Volumes | | Volumes | PLP-Car after |
|---|---|---|---|---|
| | of solvent | Anti- | of anti- | Crystallization |
| Solvent | (ml/g) | solvent | solvent (ml/g) | (%) |
| n-propanol | 30 | water | 50 | 0.05 |
| dichloromethane | 17 | hexane | 50 | 0.04 |
| dioxane | 10 | water | 50 | 0.10 |

### Example 13

### Purification of Paliperidone from PLP-Car by Filtration through Activated Carbon

A slurry of paliperidone (contaminated with 0.57% PLP-Car) in 40 volumes (g/ml) of acetone/water (3:1) was heated to reflux until complete dissolution. After the compound was dissolved, the hot solution was filtrated through hi-flow and cooled in an ice bath. The solid was filtrated and analyzed, wherein the analytical results are as shown in the next table.

| | PLP-Car after |
|---|---|
| Type of active carbon | Crystallization (%) |
| HB ultra | N/D |
| CGP super | N/D |
| GBG | N/D |
| SX plus | N/D |
| ROX 0.8 | 0.10 |
| A super eur | 0.05 |

What is claimed is:

1. Paliperidone comprising less than about 0.1% of impurity X.

2. Paliperidone of claim 1 comprising less than about 0.05% of the impurity X.

3. Paliperidone of claim 1 comprising less than about 0.02% of the impurity X.

4. Paliperidone of claim 1, comprising X at less than about 0.1%.

5. Paliperidone of claim 4, comprising X at less than about 0.05%.

6. Paliperidone of claim 5 comprising X at less than about 0.02%.

7. Paliperidone of any one of claims 4-6, further comprising PLP-Car at less than about 0.2%.

8. Paliperidone of any one of claims 4-6, further comprising PLP-Car at less than about 0.1%.

9. Paliperidone of any one of claims 4-6, further comprising PLP-Car at less than about 0.05%.

10. Paliperidone of any one of claims 4-6, further comprising PLP-Car at less than about 0.02%.

11. Paliperidone having a total purity of at least about 98% comprising impurity X at less than about 0.1%.

12. Paliperidone of claim 11 having a total purity of at least about 99%.

13. Paliperidone of claim 12 having a total purity of at least about 99.9%.

14. Paliperidone of claim 11 having a total purity of about 98% to about 99.9%.

15. Paliperidone of claim 14 having a total purity of about 99% to about 99.9%.

16. Paliperidone of claim 11 having a total purity of about 98% to about 99.99%.

17. Paliperidone of claim 16 having a total purity of about 99% to about 99.99%.

18. Paliperidone of claim 11, comprising the impurity X at less than about 0.05%.

19. Paliperidone of claim 18, comprising the impurity X at less than about 0.02%.

20. Paliperidone of claim 18, further comprising PLP-car at less than about 0.2%.

21. Paliperidone of claim 19, further comprising PLP-car at less than about 0.02%.

22. The paliperdone of claim 1 prepared by a process comprising
crystallizing paliperidone from at least one solvent selected from the group consisting of C₃₋₆ ketones, a mixture of a C₃₋₆ ketone and water, N-methylpyrrolidone, C₃₋₆ amides, halo-substituted C₆₋₁₂ aromatic hydrocarbons, propylene glycol, dimethyl sulfoxide, di-methyl carbonate, C₁₋₄ alkyl alcohols, a mixture of a C₁₋₄ alkyl alcohol and water, acetonitrile, a mixture of acetonitrile and water, C₂₋₆ alkyl acetates, a mixture of a C₂₋₆ alkyl acetate and water, cellosolve, dimethyl carbonate, polyethylene glycol methyl ether and C₂₋₈ ethers to obtain the paliperidone of claim 1.

23. The paliperidone of claim 1 prepared by a process comprising crystallizing paliperidone from a solvent, wherein the crystallizing step comprises
dissolving the paliperidone in the solvent to obtain a solution;
mixing the solution with an anti-solvent to form a mixture in order to induce crystallization of paliperidone as the paliperidone of claim 1.

24. The paliperidone of claim 1 prepared by a process comprising
(a) providing a paliperidone solution containing more than about 0.1% X;
(b) admixing the solution with finely powdered carbon; and
(c) filtrating the admixture obtained from step (b) to obtain the paliperidone of claim 1.
